(19) United States
(12) Patent Application Publication
(10) Pub. No.: US 2019/0062264 A1
(43) Pub. Date: Feb. 28, 2019

(54) ETHERAMINES BASED ON 1,3-DIALCOHOLS

(71) Applicant: BASF SE, LUDWIGSHAFEN AM RHEIN (DE)

(72) Inventors: Bjoern Ludolph, Ludwigshafen (DE); Sophia Ebert, Ludwigshafen (DE); Christian Eidamshaus, Ludwigshafen (DE); Stefano Scialla, Cincinnati, OH (US); Brian Joseph Loughnane, Cincinnati, OH (US); Karie Marie Henke, Cincinnati, OH (US); Jay Frank Nash, Cincinnati, OH (US); Michael Patrick Hayes, Cincinnati, OH (US)

(21) Appl. No.: 16/087,236

(22) PCT Filed: Mar. 14, 2017
(86) PCT No.: PCT/EP2017/055942

§ 371 Date: Sep. 21, 2018

(30) Foreign Application Priority Data
Mar. 24, 2016 (EP) 16162157.8

(51) Int. Cl. C07C 217/08; C07C 213/02

(52) U.S. Cl. CPC C07C 217/08; C08G 59/5006; C07C 213/02

(57) ABSTRACT

An etheramine mixture comprising of at least one amine selected from the group consisting of amine of Formula (I) and amine of Formula (II) wherein R₁-R₁₂ are independently selected from H, alkyl, cycloalkyi, aryl, alkylaryl, or arylalkyi, wherein at least one of R₁-R₆ and at least one of R₇-R₁₂ is different from H, and wherein Z₁-Z₃ are linear CH₂CH₂CH₂NH₂.

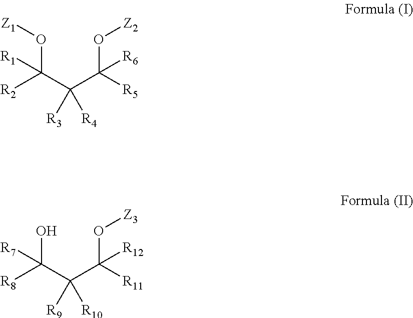

This invention relates to etheramines with linear propylamine groups based on 1,3-dialcohols.

Due to the increasing popularity of easy-care fabrics made of synthetic fibers as well as the ever increasing energy costs and growing ecological concerns of detergent users, the once popular hot water wash has now taken a back seat to washing fabrics in cold water. Many commercially available laundry detergents are even advertised as being suitable for washing fabrics at 40° C. or 30° C. or even at room temperature. To achieve satisfactory washing result at such low temperatures, results comparable to those obtained with hot water washes, the demands on low-temperature detergents are especially high.

It is known to include certain additives in detergent compositions to enhance the detergent power of conventional surfactants so as to improve the removal of grease stains at temperatures of 60° C. and below.

WO 86/07603 discloses that detergent composition comprising an aliphatic amine compound, in addition to at least one synthetic anionic and/or nonionic surfactant, are known and have led to improved cleaning results even at low wash temperatures.

Also, the use of linear, alkyl-modified (secondary) alkoxypropylamines in laundry detergents to improve cleaning at low temperatures is known (WO90/03423). These known laundry detergents, however, are unable to achieve satisfactory cleaning when laundry is washed at cold temperatures.

Furthermore, the use of linear, primary polyoxyalkyleneamines (e.g., Jeffamine® D-230) to stabilize fragrances in laundry detergents and provide longer lasting scent is also known (WO2009/065738). Also, the use of high-moleculer-weight (molecular weight of at least about 1000), branched, trifunctional, primary amines (e.g., Jeffamine® T-5000 polyetheramine) to suppress suds in liquid detergents is known (WO01/76729).

Additionally, WO 2011/087793 reads on etheramine mixtures comprising at least 10 wt % of an alkoxylated monoether amine based on polyhydric alcohols containing 2 to 4 hydroxyl groups as the starting compound. A process for the manufacture of these etheramine mixtures is also disclosed. These products find an application as a curing agent or as a raw material in the synthesis of polymers.

Furthermore, WO 2014/154783 discloses polyetheramines, wherein at least half of the terminal groups are amine groups, based on 1,3-dialcohols and their use in cleaning compositions.

There is a continuous need for cleaning compositions that remove grease stains from fabrics and other soiled materials, as grease stains are challenging stains to remove. Conventional cleaning compositions directed to grease removal frequently utilize various amine compounds which tend to show strong negative impacts on whiteness. As a consequence there is still a continual need for improved amine compositions which provide improved grease removal from fabrics and other soiled materials and at the same time do not negatively impact the clay cleaning. There is also a need for compounds which would improve the washing performance of detergents at low temperatures, e.g. at temperatures as low as 30° C. or even lower.

It was the object of the present invention to comply with such needs.

This goal was achieved by the present invention as described herein below and as reflected in the claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

The present invention relates to an etheramine mixture comprising of at least one amine selected from the group consisting of amine of Formula (I) and amine of Formula (II),

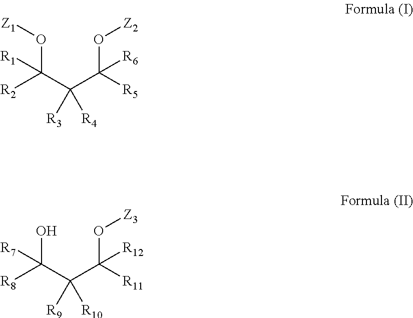

wherein R₁-R₁₂ are independently selected from H, alkyl, cycloalkyl, aryl, alkylaryl, or arylalkyl, wherein at least one of R₁-R₆ and at least one of R₇-R₁₂ is different from H, and wherein Z₁-Z₃ are linear CH₂CH₂CH₂NH₂. Optionally the diol of Formula (III) may be comprised in the etheramine mixture.

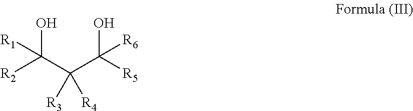

The etheramine mixture according to the present invention of Formula (I) and (II) comprising linear propylamine groups (CH₂CH₂CH₂NH₂) provide improved washing performance of detergents.

In one embodiment of the present invention, the etheramine mixture may comprise at least 90% by weight, based on the total weight of the etheramine mixture, of the amine of Formula (I) and/or (II). In one embodiment of the present invention, the etheramine mixture may comprise at least 95% by weight, based on the total weight of the etheramine mixture, of the amine of Formula (I) and/or (II).

In Formula (I) or (II), R₁, R₂, R₅, R₆, R₇, R₈, R₁₁, and R₁₂ may be H, and R₃, R₄, R₉, and R₁₀ may independently be selected from C₁-₁₆ alkyl and aryl.

In one embodiment of the present invention, in Formula (I) or (II), R₁, R₂, R₅, R₆, R₇, R₈, R₁₁, and R₁₂ may be H, and R₃, R₄, R₉, and R₁₀ may independently be selected from a butyl group, an ethyl group, a methyl group, a propyl group, and a phenyl group.

In one specific embodiment of the present invention, in Formula (I) or (II), R₃ and R₉ may be each an ethyl group, R₁, R₂, R₅, R₆, R₇, R₈, R₁₁, and R₁₂ may be each H, and/or R₄ and R₁₀ may be each a butyl group .

The etheramine of Formula (I) or Formula (II) may have a weight average molecular weight of from 150 to 1000 grams/mol, or of from 200 to 500 grams/mol, or of from 300 to about 450 grams/mol.

The etheramine mixture comprising of at least one amine selected from the group consisting of amine of Formula (I) and amine of Formula (II) wherein Z₁-Z₃ are linear CH₂CH₂CH₂NH₂ may be obtainable by reductive cyanoethylation of 1,3-diols of formula (III).

Generally, as used herein, the term “obtainable by” means that corresponding products do not necessarily have to be produced (i.e. obtained) by the corresponding method or process described in the respective specific context, but also products are comprised which exhibit all features of a product produced (obtained) by said corresponding method or process, wherein said products were actually not produced (obtained) by such method or process. However, the term “obtainable by” also comprises the more limiting term “obtained by”, i.e. products which were actually produced (obtained) by a method or process described in the respective specific context.

In one embodiment of the present invention, in the 1,3-diol of Formula (III) R₁, R₂, R₅, R₆ are H and R₃, R₄ may be C1-16 alkyl or aryl.

The 1,3-diol of Formula (III) may be selected from the group consisting of 2-butyl-2-ethyl-1,3-propanediol, 2-methyl-2-propyl-1,3-propanediol, 2-methyl-2-phenyl-1,3-propanediol, 2,2-dime-thyl-1,3-propanediol, and 2-ethyl-1,3-hexanediol.

Amination of the 1,3-diols may be carried out by reductive cyanoethylation, and leads to new structures with Formula I and/or (II):

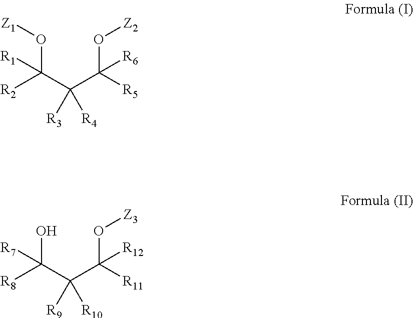

wherein R₁-R₁₂ are independently selected from H, alkyl, cycloalkyl, aryl, alkylaryl, or arylalkyl, wherein at least one of R₁-R₆ and at least one of R₇-R₁₂ is different from H, and wherein Z₁-Z₃ are linear CH₂CH₂CH₂NH₂.

Optionally, the diol of Formula (III) may be comprised in the mixture as well.

The reductive cyanoethylation may be carried out by reaction of the 1,3-diol mixture (Formula III) with acrylonitrile in the presence of a base followed by hydrogenation with hydrogen and a catalyst. The use of acrylonitrile leads to linear propylamine end groups according to the present invention.

Suitable bases typically comprise alkaline hydroxides, and substituted ammonium hydroxide. Preferably, tetrakis(2-hydroxyethyl)ammonium hydroxide is used as a base.

As catalysts for hydrogenation the nitrile function to the corresponding amine, it is possible to use, in particular, catalysts which comprise one or more elements of the 8^{th} transition group of the Periodic Table (Fe, Co, Ni, Ru, Rh, Pd, Os, Ir, Pt), preferably Fe, Co, Ni, Ru or Rh, particularly preferably Co or Ni, in particular Co, as active component. A further preferred active component is Cu.

The abovementioned catalysts can be doped in the usual way with promoters, for example chromium, iron, cobalt, manganese, molybdenum, titanium, tin, metals of the alkali metal group, metals of the alkaline earth metal group and/or phosphorus.

As catalysts, preference can be given to using skeletal catalysts (also referred to as Raney® type, hereinafter also: Raney catalyst) which are obtained by leaching (activating) an alloy of hydrogenation-active metal and a further component (preferably Al). Preference is given to using Raney nickel catalysts or Raney cobalt catalysts.

Furthermore, supported Pd or Pt catalysts are preferably used as catalysts. Preferred support materials are activated carbon, Al20 3, Ti02, Zr02 and Si02. In a very preferred embodiment, catalysts produced by reduction of catalyst precursors are used in the process of the invention.

The catalyst precursor comprises an active composition which comprises one or more catalytically active components, optionally promoters and optionally a support material.

The catalytically active components comprise oxygen-comprising compounds of the above-mentioned metals, for example the metal oxides or hydroxides thereof, e.g. CoO, NiO, CuO and/or mixed oxides thereof.

For the purposes of the present patent application, the term “catalytically active components” is used for abovementioned oxygen-comprising metal compounds but is not intended to apply that these oxygen-comprising compounds are themselves catalytically active. The catalytically active components generally display catalytic activity in the reaction according to the invention only after reduction.

Particular preference is given to catalyst precursors such as the oxide mixtures which are disclosed in EP-A-0636409 and before reduction with hydrogen comprise from 55 to 98% by weight of Co, calculated as CoO, from 0.2 to 15% by weight of phosphorus, calculated as H₃PO₄, from 0.2 to 15% by weight of manganese, calculated as MnO₂, and from 0.2 to 5.0% by weight of alkali metal, calculated as M₂O (M=alkali metal), or oxide mixtures which are disclosed in EP-A-0742045 and before reduction with hydrogen comprise from 55 to 98% by weight of Co, calculated as CoO, from 0.2 to 15% by weight of phosphorus, calculated as H₃PO₄, from 0.2 to 15% by weight of manganese, calculated as MnO₂, and from 0.05 to 5% by weight of alkali metal, calculated as M₂O (M=alkali metal).

Alternatively, sponge type catalysts of cobalt and nickel can be used.

The process can be carried out in a continuous or discontinuous mode, e.g. in an autoclave, tube reactor or fixed-bed reactor. The reactor design is also not narrowly critical. The feed thereto may be upflowing or downflowing, and design features in the reactor which optimize plug flow in the reactor may be employed.

In one embodiment of the present invention, the degree of amination of the etheramine mixture comprising of at least one amine selected from the group consisting of amine of Formula (I) and amine of Formula (II) is equal to or greater than 50%. In another embodiment of the present invention, the degree of amination is equal to or greater than 55%. In another embodiment the degree of amination is in the range of from 60 to 95%. In a further embodiment the degree of amination is in the range of from 65 to 90%. In another embodiment the degree of amination is in the range of from 70 to 85%.

Unless specified otherwise herein, the degree of amination is calculated from the total amine value (AZ) divided by sum of the total acetylables value (AC) and tertiary amine value(tert. AZ) multiplied by 100:

(Total AZ: (AC+tert. AZ)×100).

The total amine value (AZ) is determined according to DIN 53176.

The total acetylables value (AC) is determined according to DIN 53240.

The secondary+tertiary amine value is determined according to ASTM D2074.

The tertiary amine value is determined according to ASTM D2074.

The primary amines value is calculated as follows: primary amine value=AZ−secondary+tertiary amine value.

Primary amine in % of total amine is calculated as follows:

Primary amine in %=((AZ−secondary+tertiary amine value)/AZ)*100

The hydroxyl value is calculated from (total acetylables value+tertiary amine value)−total amine value.

In another preferred embodiment, the etheramines of the invention can also be further reacted with an acid. The acid may be selected from the group consisting of citric acid, lactic acid, sulfuric acid, methanesulfonic acid, hydrogen chloride, phosphoric acid, formic acid, acetic acid, propionic acid, valeric acid, oxalic acid, succinic acid, adipic acid, sebacic acid, glutaric acid, glucaric acid, tartaric acid, malic acid, benzoic acid, salicylic acid, phthalic acid, oleic acid, stearic acid, caproic acid, caprylic acid, capric acid, lauric acid, myristic acid, palmitic acid, linoleic acid and mixtures thereof. In another embodiment the acid may be selected from the group consisting of caproic acid, caprylic acid, capric acid, lauric acid, and myristic acid. In an alternative embodiment, the etheramines of the invention may, in protonated form, have a surfactant as a counter ion, as obtained from e.g. linear alkyl benzene sulphonic acid.

Applications:

The inventive etheramine mixtures obtained by reductive cyanoethylation may be used in personal care, especially in shampoo and body wash formulations.

They may also be used as curing agent for epoxy resins or as a reactant in the production of polymers but also in polyurethanes, polyureas, epoxy resins, polyamides.

The inventive etheramines have proved to be effective for removal of stains, particularly grease, from soiled material. Besides, cleaning compositions with inventive etheramines also do not have the cleaning negatives seen with conventional, amine cleaning compositions for hydrophilic bleachable stains, such as coffee, tea, wine, or particulates. Additionally, for stain removal from white fabric, cleaning compositions with inventive etheramines do not cause the whiteness negatives that commercially available, amine cleaning compositions cause.

A further advantage of cleaning compositions comprising the inventive etheramines is their ability to remove grease stains in cold water cleaning solutions, via pretreatment of the grease stain outside the washing machine, followed by cold water washing. Without being limited by theory, cold water solutions have the effect of causing greases to harden or solidify, making greases more resistant to removal, especially from fabric. Cleaning compositions with with etheramine mixtures according to Formula (I) and/or (II) linear propylamine groups (CH₂CH₂CH₂NH₂) however, are surprisingly effective when used in pretreatment followed by cold water cleaning.

As used herein the phrase “cleaning composition” includes compositions and formulations designed for cleaning soiled material. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, liquid hand dish-washing composition, detergent contained on or in a porous substrate or nonwoven sheet, automatic dish-washing agent, hard surface cleaner, and other suitable forms that may be apparent to one skilled in the art in view of the teachings herein. Such compositions may be used as a pre-laundering treatment, a post-laundering treatment, may be added during the rinse or wash cycle of the laundering operation, or used in homecare cleaning applications. The cleaning compositions may have a form selected from liquid, powder, single-phase or multi-phase unit dose, pouch, tablet, gel, paste, bar, or flake.

The cleaning compositions described herein may include from about 0.1% to about 10%, in some examples, from about 0.2% to about 5%, and in other examples, from about 0.5% to about 3%, by weight the composition, of an amine-terminated diol of Formula I and/or II.

The inventive etheramine mixtures are effective for removal of stains, particularly grease, from soiled material. Cleaning compositions containing the amine-terminated diols of the invention also do not exhibit the cleaning negatives seen with conventional amine-containing cleaning compositions on hydrophilic bleachable stains, such as coffee, tea, wine, or particulates. Additionally, unlike conventional amine-containing cleaning compositions, the amine-terminated diols of the invention do contribute less to whiteness negatives on white fabrics compared to conventional amine-containing cleaning compositions.

A further advantage of cleaning compositions containing the inventive etheramine mixture is their ability to remove grease stains in cold water, for example, via pretreatment of a grease stain followed by cold water washing. Without being limited by theory, it is believed that cold water washing solutions have the effect of hardening or solidifying grease, making the grease more resistant to removal, especially on fabric. Cleaning compositions containing etheramins with linear propylamine groups (CH₂CH₂CH₂NH₂) of the invention are surprisingly effective when used as part of a pretreatment regimen followed by cold water washing.

Surfactant System

The cleaning compositions comprise a surfactant system in an amount sufficient to provide desired cleaning properties. In some embodiments, the cleaning composition comprises, by weight of the composition, from about 1% to about 70% of a surfactant system. In other embodiments, the liquid cleaning composition comprises, by weight of the composition, from about 2% to about 60% of the surfactant system. In further embodiments, the cleaning composition comprises, by weight of the composition, from about 5% to about 30% of the surfactant system. The surfactant system may comprise a detersive surfactant selected from anionic surfactants, nonionic surfactants, cationic surfactants, zwitterionic surfactants, amphoteric surfactants, ampholytic surfactants, and mixtures thereof. Those of ordinary skill in the art will understand that a detersive surfactant encompasses any surfactant or mixture of surfactants that provide cleaning, stain removing, or laundering benefit to soiled material.

Adjunct Cleaning Additives

The cleaning compositions of the invention may also contain adjunct cleaning additives. Suitable adjunct cleaning additives include builders, structurants or thickeners, clay soil removal/anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, hueing agents, dye transfer inhibiting agents, chelating agents, suds supressors, softeners, and perfumes.

Methods of Use

The present invention includes methods for cleaning soiled material. As will be appreciated by one skilled in the art, the cleaning compositions of the present invention are suited for use in laundry pretreatment applications, laundry cleaning applications, and home care applications.

Such methods include, but are not limited to, the steps of contacting cleaning compositions in neat form or diluted in wash liquor, with at least a portion of a soiled material and then optionally rinsing the soiled material. The soiled material may be subjected to a washing step prior to the optional rinsing step.

For use in laundry pretreatment applications, the method may include contacting the cleaning compositions described herein with soiled fabric. Following pretreatment, the soiled fabric may be laundered in a washing machine or otherwise rinsed.

Machine laundry methods may comprise treating soiled laundry with an aqueous wash solution in a washing machine having dissolved or dispensed therein an effective amount of a machine laundry cleaning composition in accord with the invention. An “effective amount” of the cleaning composition means from about 20 g to about 300 g of product dissolved or dispersed in a wash solution of volume from about 5 L to about 65 L. The water temperatures may range from about 5° C. to about 100° C. The water to soiled material (e.g., fabric) ratio may be from about 1:1 to about 20:1. In the context of a fabric laundry composition, usage levels may also vary depending not only on the type and severity of the soils and stains, but also on the wash water temperature, the volume of wash water, and the type of washing machine (e.g., top-loading, front-loading, top-loading, vertical-axis Japanese-type automatic washing machine).

The cleaning compositions herein may be used for laundering of fabrics at reduced wash temperatures. These methods of laundering fabric comprise the steps of delivering a laundry cleaning composition to water to form a wash liquor and adding a laundering fabric to said wash liquor, wherein the wash liquor has a temperature of above 0° C. to about 20° C., or to about 15 ° C., or to about 10 ° C. The fabric may be contacted to the water prior to, or after, or simultaneous with, contacting the laundry cleaning composition with water.

Another method includes contacting a nonwoven substrate impregnated with an embodiment of the cleaning composition with soiled material. As used herein, “nonwoven substrate” can comprise any conventionally fashioned nonwoven sheet or web having suitable basis weight, caliper (thickness), absorbency, and strength characteristics. Non-limiting examples of suitable commercially available nonwoven substrates include those marketed under the tradenames SONTARA® by DuPont and POLYWEB® by James River Corp.

Hand washing methods, and combined handwashing with semiautomatic washing machines, are also included.

Machine Dishwashing Methods

Methods for machine-dishwashing or hand dishwashing soiled dishes, tableware, silverware, or other kitchenware, are included. One method for machine dishwashing comprises treating soiled dishes, tableware, silverware, or other kitchenware with an aqueous liquid having dissolved or dispensed therein an effective amount of a machine dishwashing composition in accord with the invention. By an effective amount of the machine dishwashing composition it is meant from about 8 g to about 60 g of product dissolved or dispersed in a wash solution of volume from about 3 L to about 10 L.

One method for hand dishwashing comprises dissolution of the cleaning composition into a receptacle containing water, followed by contacting soiled dishes, tableware, silverware, or other kitchenware with the dishwashing liquor, then hand scrubbing, wiping, or rinsing the soiled dishes, tableware, silverware, or other kitchenware. Another method for hand dishwashing comprises direct application of the cleaning composition onto soiled dishes, tableware, silverware, or other kitchenware, then hand scrubbing, wiping, or rinsing the soiled dishes, tableware, silverware, or other kitchenware. In some examples, an effective amount of cleaning composition for hand dishwashing is from about 0.5 ml. to about 20 ml diluted in water.

Packaging for the Compositions

The cleaning compositions described herein can be packaged in any suitable container including those constructed from paper, cardboard, plastic materials, and any suitable laminates. An optional packaging type is described in European Application No. 94921505.7.

Multi-Compartment Pouch Additive

The cleaning compositions described herein may also be packaged as a multi-compartment cleaning composition.

The present invention is further demonstrated and exemplified in the following examples, however, without being limited to the embodiments described in the examples.

### EXAMPLES

¹H-NMR and ¹³C-NMR measurements were carried out in CDCl₃ with a Bruker 400 MHz spectrometer.

Unless specified otherwise herein, the degree of amination is calculated from the total amine value (AZ) divided by sum of the total acetylables value (AC) and tertiary amine value(tert. AZ) multiplied by 100:

(Total AZ:(AC+tert. AZ)×100).

The total amine value (AZ) is determined according to DIN 53176.

The total acetylables value (AC) is determined according to DIN 53240.

The secondary+tertiary amine value is determined according to ASTM D2074.

The tertiary amine value is determined according to ASTM D2074.

The primary amines value is calculated as follows: primary amine value=AZ−secondary+tertiary amine value.

Primary amine in % of total amine is calculated as follows:

Primary amine in %=((AZ−secondary+tertiary amine value)/AZ)*100

The hydroxyl value is calculated from (total acetylables value+tertiary amine value)−total amine value.

All percentages are presented as percentage based on weight unless otherwise indicated.

### Example 1a

### 1 mol 2-butyl-2-ethyl-1,3-propanediol+2.0 mol acrylonitrile

In a 4-neck glass vessel with reflux condenser, nitrogen inlet, thermometer, and dropping funnel 216.3 g molten 2-butyl-2-ethyl-1,3-propanediol and 3.1 g tetrakis(2-hydroxyethyl)ammonium hydroxide (50% in water) was charged at 50° C. The temperature was increased to 60° C. and 171.9 g acrylonitrile was added dropwise within 1.0 h. During the addition the temperature was allowed to rise to 70° C. The reaction mixture was stirred at 60° C. for 3 h and filtered and volatile compounds were removed in vacuo. 353.0 g of a orange liquid was obtained. ¹H-NMR in CDCl₃ showed complete conversion of acrylonitrile.

### Example 1b

### 1 mol 2-butyl-2-ethyl-1,3-propanediol+2.0 mol acrylonitrile, hydrogenated

The nitrile was continuously hydrogenated in a tubular reactor (length 500 mm, diameter 18 mm) filled with a splitted cobalt catalyst prepared as described in EP636409. At a temperature of 110° C. and a pressure of 160 bar, 15.0 g of a solution of the nitrile in THF (20%), 24.0 g of ammonia and 16 norm litre (NL) of hydrogen were passed through the reactor per hour. The crude material was collected and stripped on a rotary evaporator to remove excess ammonia, light weight amines and THF to afford the hydrogenated product. ¹H and ¹³C-NMR analysis showed full conversion of the nitrile. The analytical data by means of titration is summarized in table 1.

TABLE 1

| Total | | | Tertiary | | Primary |
|---|---|---|---|---|---|
| amine- | Total | Secondary | amine- | | Amine |
| value | acetylables | and tertiary | value | Degree of | value |
| mg | value | amine | mg | amination | in % of total |
| KOH/g | mg KOH/g | mg KOH/g | KOH/g | in % | amine |
| 408.3 | 408.7 | 1.87 | 1.78 | 99.5 | 99.5 |

### Example 2a

### 1 mol 2-butyl-2-ethyl-1,3-propanediol+1.2 mol acrylonitrile

In a 4-neck glass vessel with reflux condenser, nitrogen inlet, thermometer, and dropping funnel 240.4 g molten 2-butyl-2-ethyl-1,3-propanediol and 3.5 g tetrakis(2-hydroxyethyl)ammonium hydroxide (50% in water) was charged at 50° C. The temperature was increased to 60° C. and 95.5 g acrylonitrile was added dropwise within 1.0 h at 60-70° C. The reaction mixture was stirred at 60° C. for 3 h and filtered and volatile compounds were removed in vacuo. 372.0 g of a light yellow liquid was obtained. ¹H-NMR in CDCl₃ showed complete conversion of acrylonitrile.

### Example 2b

### 1 mol 2-butyl-2-ethyl-1,3-propanediol+1.2 mol acrylonitrile, hydrogenated

The nitrile was continuously hydrogenated in a tubular reactor (length 500 mm, diameter 18 mm) filled with a splitted cobalt catalyst prepared as described in EP636409. At a temperature of 110° C. and a pressure of 160 bar, 15.0 g of a solution of the nitrile in THF (20%), 24.0 g of ammonia and 16 NL of hydrogen were passed through the reactor per hour. The crude material was collected and stripped on a rotary evaporator to remove excess ammonia, light weight amines and THF to afford the hydrogenated product. ¹H and ¹³C-NMR analysis showed full conversion of the nitrile. The analytical data by means of titration is summarized in table 2.

TABLE 2

| Total | | | Tertiary | | |
|---|---|---|---|---|---|
| amine- | Total | Secondary | amine- | | Primary |
| value | acetylables | and tertiary | value | Degree of | Amine value |
| mg | value | amine | mg | amination | in % of total |
| KOH/g | mg KOH/g | mg KOH/g | KOH/g | in % | amine |
| 278.8 | 501.5 | 1.8 | 1.4 | 55.4 | 99.4 |

### Example 3a

### 1 mol 2-ethyl-1,3-hexanediol+2.0 mol acrylonitrile

In a 4-neck glass vessel with reflux condenser, nitrogen inlet, thermometer, and dropping funnel 197.4 g 2-ethyl-1,3-hexanediol and 3.2 g tetrakis(2-hydroxyethyl)ammonium hydroxide (50% in water) was charged at 50° C. The temperature was increased to 60° C. and 186.2 g acrylonitrile was added dropwise within 1.0 h at 60-70° C. The reaction mixture was stirred at 60° C. for 3 h and filtered and volatile compounds were removed in vacuo. 375.0 g of a dark yellow liquid was obtained. ¹H-NMR in CDCl₃ showed complete conversion of acrylonitrile.

### Example 3b

### 1 mol 2-ethyl-1,3-hexanediol+2.0 mol acrylonitrile, hydrogenated

The nitrile was continuously hydrogenated in a tubular reactor (length 500 mm, diameter 18 mm) filled with a splitted cobalt catalyst prepared as described in EP636409. At a temperature of 110° C. and a pressure of 160 bar, 15.0 g of a solution of the nitrile in THF (20%), and 16 NL of hydrogen were passed through the reactor per hour. The crude material was collected and stripped on a rotary evaporator to remove excess ammonia, light weight amines and THF to afford the hydrogenated product. ¹H and ¹³C-NMR analysis showed full conversion of the nitrile. The analytical data by means of titration is summarized in table 3.

TABLE 3

| Total | | | Tertiary | | |
|---|---|---|---|---|---|
| amine- | Total | Secondary | amine- | | Primary |
| value | acetylables | and tertiary | value | Degree of | Amine value |
| mg | value | amine | mg | amination | in % of total |
| KOH/g | mg KOH/g | mg KOH/g | KOH/g | in % | amine |
| 376.4 | 471.3 | 15.8 | 1.8 | 79.6 | 95.8 |

### Comparative Example 1a

### 1 mol 2-butyl-2-ethyl-1,3-propandiol+5.6 mol propylene oxide

In a 2 I autoclave 1286.7 g 2-Butyl-2-ethyl-1,3-propane diol and 15.5 g KOH (50% in water) were mixed and stirred under vacuum (<10 mbar) at 90° C. for 2 h. The autoclave was purged with nitrogen and heated to 140° C. 2612.0 g propylene oxide was added within 26 h. To complete the reaction, the mixture was allowed to post-react for additional 10 h at 140° C. The reaction mixture was stripped with nitrogen and volatile compounds were removed in vacuo at 80° C. The catalyst was removed by adding 211.0 g water and 33.9 g phosphoric acid (40% in water) stirring at 100° C. for 0.5 h and dewatering in vacuo for 2 hours. After filtration 3901.0 g of a light yellowish oil was obtained (hydroxy value: 190 mgKOH/g).

### Comparative Example 1b

### 1 mol 2-butyl-2-ethyl-1,3-propandiol+5.6 mol propylene oxide, aminated

The amination of 2-butyl-2-ethyl-1,3-propanediol+2,8 PO/OH (1) was conducted in a tubular reactor (length 500 mm, diameter 18 mm) which had been charged with 15 mL of silica (3×3 mm pellets) followed by 70 mL (74 g) of the catalyst precursor (containing oxides of nickel, cobalt, copper and tin on gama-Al₂O₃, 1,0-1,6 mm split—prepared according to WO 2013/072289 A1) and filled up with silica (ca. 15 mL).

After catalyst activation the alcohol was aminated at a WHSV of 0.19 kg/liter*h (molar ratio ammonia/alcohol=55:1, hydrogen/alcohol=11,6:1) at 206° C. The crude material was collected and stripped on a rotary evaporator to remove excess ammonia, light weight amines and reaction water to afford aminated 1. The analytical data of the reaction product is shown below.

| Total | | Secondary | Tertiary | | | Primary |
|---|---|---|---|---|---|---|
| amine- | Total | and tertiary | amine- | Hydroxyl | Degree of | Amine value |
| value | acetylatables | amine value | value | value | amination | in % of total |
| mg KOH/g | mg KOH/g | mg KOH/g | mg KOH/g | mg KOH/g | in % | amine |
| 222.92 | 231.50 | 2.57 | 0.31 | 8.89 | 96.16 | 98.85 |

2. Use as Additives in Laundry Detergent

Technical stain swatches of blue knitted cotton containing Beef Fat, Pork Fat, Chicken Fat and Bacon Grease were purchased from Warwick Equest Ltd. The stains were washed for 30 min in a launder-o-meter (manufactured by SDL Atlas) at room temperature using per canister 500 mL of washing solution, 20 steel balls (weight of 1 ball is 1 g) and ballast fabrics. The washing solution contained 5000 ppm of detergent composition DC1 (table 1). Water hardness was 2.5 mM (Ca²⁺:Mg²⁺ molar ratio was 4:1). Additives were added to the washing solution of each canister separately and in the amount as detailed below. After addition the pH value was re-adjusted to the pH value of washing solution without additive.

Standard colorimetric measurement was used to obtain L*, a* and b* values for each stain before and after the washing. From L*, a* and b* values the stain level were calculated as color difference ΔE (calculated according to DIN EN ISO 11664-4) between stain and untreated fabric. Stain removal from the swatches was calculated as follows:

$$Stain Removal Index ( SRI ) = \frac{Δ {E}_{inital} - Δ {E}_{washed}}{Δ {E}_{initial}} \times 100$$

ΔEᵢₙᵢₜᵢₐᵢₗ=Stain level before washing

ΔE_{washed}=Stain level after washing

Stain level corresponds to the amount of grease on the fabric. The stain level of the fabric before the washing (ΔEᵢₙᵢₜᵢₐᵢₗ) ishigh, in the washing process stains are removed and the stain level after washing is smaller (ΔE_{washed}). The better the stains have been removed the lower the value for ΔE_{washed} will be and the higher the difference will be to ΔEᵢₙᵢₜᵢₐₗ. Therefore, the value of stain removal index increases with better washing performance.

TABLE 4
Detergent composition DC1

| Ingredients of liquid detergent composition DC1 | percentage by weight |
|---|---|
| Alkyl Benzene sulfonate¹ | 7.50% |
| AE3S² | 2.60% |
| AE9³ | 0.40% |
| NI 45-7⁴ | 4.40% |
| Citric Acid | 3.20% |
| C1218 Fatty acid | 3.10% |
| Amphiphilic polymer⁵ | 0.50% |
| Zwitterionic dispersant⁶ | 1.00% |
| Ethoxylated Polyethyleneimine⁷ | 1.51% |
| Protease⁸ | 0.89% |
| Enymes⁹ | 0.21% |
| Chelant¹⁰ | 0.28% |
| Brightener¹¹ | 0.09% |
| Solvent | 7.35% |
| Sodium Hydroxide | 3.70% |
| Fragrance & Dyes | 1.54% |
| Water, filler, stucturant | To Balance |

¹Linear alkylbenenesulfonate having an average aliphatic carbon chain length C11-C12 supplied by Stepan, Northfield Illinois, USA
²AE3S is C12-15 alkyl ethoxy (3) sulfate supplied by Stepan, Northfield, Illinois, USA
³AE9 is C12-14 alcohol ethoxylate, with an average degree of ethoxylation of 9, supplied by Huntsman, Salt Lake City, Utah, USA
⁴NI 45-7 is C14-15 alcohol ethoxylate, with an average degree of ethoxylation of 7, supplied by Huntsman, Salt Lake City, Utah, USA
⁵Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.
⁶A compound having the following general structure: bis((C2H5O)(C2H4O)n)(CH3)—N+—CxH2x—N+—(CH3)—bis((C2H5O)(C2H4O)n), wherein n = from 20 to 30, and x = from 3 to 8, or sulphated or sulphonated variants thereof
⁷Polyethyleneimine (MW = 600) with 20 ethoxylate groups per —NH
⁸Proteases may be supplied by Genencor International, Palo Alto, California, USA (e.g. Purafect Prime ®) or by Novozymes, Bagsvaerd, Denmark (e.g. Liquanase ®, Coronase ®).
⁹Natalase ®, Mannaway ® are all products of Novozymes, Bagsvaerd, Denmark.
¹⁰Suitable chelants are, for example, diethylenetetraamine pentaacetic acid (DTPA) supplied by Dow Chemical, Midland, Michigan, USA or Hydroxyethane di phosphonate (HEDP) or diethylene triamine penta(methyl phosphonic) acid supplied by Solutia, St Louis, Missouri, USA;
¹¹Fluorescent Brightener 1 is Tinopal ® AMS, Fluorescent Brightener 2 supplied by Ciba Specialty Chemicals, Basel, Switzerland

Washing Test 1:

| | | SRI, | SRI, | SRI, | SRI, |
|---|---|---|---|---|---|
| | additive/ | Beef | Pork | Chicken | Bacon |
| Additive | [g] | Fat | Fat | Fat | Grease |
| without additive | — | 27.2 | 24.9 | 25.5 | 39.3 |
| Comparitive example 1b | 0.0375 | 41.0 | 36.9 | 40.4 | 51.0 |
| Example 1b | 0.0375 | 43.0 | 42.5 | 44.5 | 60.0 |

Washing Test 2

| | | | | SRI, | SRI, |
|---|---|---|---|---|---|
| | additive/ | SRI, | SRI, | Chicken | Bacon |
| Additive | [g] | Beef Fat | Pork Fat | Fat | Grease |
| without additive | — | 27.8 | 27.4 | 22.8 | 37.5 |
| Example 3b | 0.0375 | 36.9 | 37.3 | 34.9 | 52.2 |

1. An etheramine mixture comprising of at least one amine selected from the group consisting of amine of Formula (I) and amine of Formula (II), 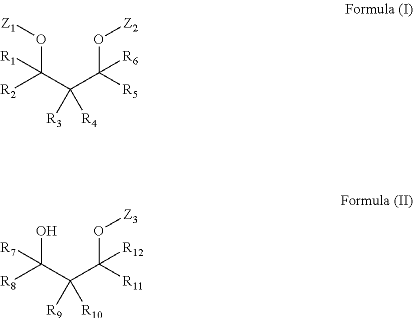
wherein R₁-R₁₂ are independently selected from H, alkyl, cycloalkyl, aryl, alkylaryl, or arylalkyl, wherein at least one of R₁-R₆ and at least one of R₇-R₁₂ is different from H, and wherein Z₁-Z₃ are linear CH₂CH₂CH₂NH₂.

2. The etheramine mixture according to claim 1, wherein the etheramine mixture comprises at least 90% by weight, based on a total weight of the etheramine mixture, of an amine of Formula (I) and/or (II).

3. The etheramine mixture according to claim 1, wherein in said amine of Formula (I) or Formula (II), R₁, R₂, R₅, R₆, R₇, R₈, R₁₁, and R₁₂ are H and R₃, R₄, R₉, and R₁₀ are independently selected from C1-16 alkyl or aryl.

4. The etheramine mixture according to claim 1, wherein in said amine of Formula (I) or Formula (II), R₁, R₂, R₅, R₆, R₇, R₈, R₁₁, and R₁₂ are H and R₃, R₄, R₉, and R₁₀ are independently selected from a butyl group, an ethyl group, a methyl group, a propyl group, or a phenyl group.

5. The etheramine mixture according to claim 1, wherein in said amine Formula (I) or Formula (II), R₃ and R₉ are each an ethyl group, R₁, R₂, R₅ R₆, R₇, R₈, R₁₁, R₁₂ are each H, R₄ and R₁₀ are each a butyl group.

6. The etheramine mixture according to claim 1, wherein the amine of Formula (I) or Formula (II) has a weight average molecular weight of about 150 to about 1000 grams/mol.

7. The etheramine mixture according to claim 1, wherein the amine of Formula (I) or Formula (II) is reacted with an acid.

8. A method of using the etheramine mixture of claim 1, the method comprising using said etheramine mixture in personal care.

9. A method of using the etheramine mixture of claim 1, the method comprising using said etheramine mixture in shampoo and body wash formulations.

10. A method of using the etheramine mixture of claim 1, the method comprising using said etheramine mixture as a curing agent for epoxy resins or as a reactant in the production of polymers.

11. A method of using the etheramine mixture of claim 1, the method comprising using said etheramine mixture in polyurethanes, polyureas, and as thermoplastic polyamide adhesives.

12. A process of production of etheramine mixtures according to claim 1 by cyanoethylation of a diol of Formula (III), 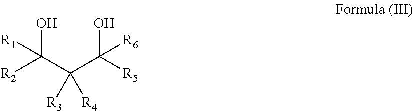
wherein R₁-R₆ are independently selected from H, alkyl, cycloalkyl, aryl, alkylaryl, or arylalkyl.
